(19) United States
(12) Patent Application Publication
(10) Pub. No.: US 2023/0111975 A1
(43) Pub. Date: Apr. 13, 2023

(54) Wearable Device, Perspiration Analysis Device, and Perspiration Analysis Method

(71) Applicant: Nippon Telegraph and Telephone Corporation, Tokyo (JP)

(72) Inventors: Yuki Hashimoto, Tokyo (JP); Takako Ishihara, Tokyo (JP); Kei Kuwabara, Tokyo (JP)

(21) Appl. No.: 17/801,988

(22) PCT Filed: Mar. 4, 2020
(86) PCT No.: PCT/JP2020/009100

§ 371 Date: Aug. 24, 2022

(51) Int. Cl. A61B 5/145; A61B 5/00

(52) U.S. Cl. CPC A61B 5/14517; A61B 5/4266

(57) ABSTRACT

A wearable device attached to a living body includes a substrate that forms a first flow path, a second flow path, and a third flow path, a light source that is disposed on the substrate and emits light toward the second flow path, and a light receiving element that is disposed on the substrate to face the light source, receives the light emitted from the light source and transmitted through the second flow path, converts the received light into an electrical signal, and outputs the electrical signal.

### CROSS-REFERENCE TO RELATED APPLICATIONS

This application is a national phase entry of PCT Application No. PCT/JP2020/009100, filed on Mar. 4, 2020, which application is hereby incorporated herein by reference.

### TECHNICAL FIELD

The present disclosure relates to a wearable device, a perspiration analysis apparatus, and a perspiration analysis method.

### BACKGROUND

A living body such as a human body has tissues that perform electrical activities such as muscles and nerves, and in order to keep these tissues operating normally, it is provided with a mechanism for keeping an electrolyte concentration in the body constant mainly by the actions of the autonomic nervous system and the endocrine system.

For example, when a human body is exposed to a hot environment for an extended period of time, and excessive exercise or the like is taken, a large amount of moisture in the body is lost due to perspiration, and an electrolyte concentration may fall outside a normal value. In such a case, various symptoms typified by heatstroke occur in the human body. Thus, in order to recognize a dehydration condition of the body, it can be said that monitoring an amount of perspiration and an electrolyte concentration in sweat is one of beneficial techniques.

For example, in NPL 1, as a typical related art for measuring an amount of perspiration, a change in an amount of water vapor during perspiration is measured. In the technique described in NPL 1, an amount of perspiration is estimated based on a difference in humidity with respect to the outside air, and thus the air in a measurement system needs to be replaced by using an air pump.

Then, in recent years, wearable devices attached to a user are becoming widespread due to development of the ICT industry and a reduction in size and weight of a computer. The wearable devices are attracting attention for practical use in health care and fitness fields.

For example, even when a measurement technique for monitoring an amount of perspiration of a user and an electrolyte concentration in sweat is implemented by a wearable device, it is necessary to reduce the size of the device. For example, when the technique for measuring an amount of perspiration described in NPL 1 is to be implemented by a wearable device, an air pump for replacing the air in a measurement system occupies relatively large volume, and thus it can be said that a reduction in size of the entire device has a problem.

### CITATION LIST

### Non Patent Literature

NPL 1: Noriko Tsuruoka, Takahiro Kono, Tadao Matsunaga, Ryoichi Nagatomi, Yoichi Haga, “Development of Small Sweating Rate Meters and Sweating Rate Measurement during Mental Stress Load and Heat Load”, Transactions of Japanese Society for Medical and Biological Engineering, Vol. 54, No. 5, pp. 207-217, 2016.

### SUMMARY

### Technical Problem

The present disclosure has been made to solve the above-described problems, and an object thereof is to provide a wearable device that can measure a physical amount of sweat without using an air pump for replacing the air in a measurement system.

### Means for Solving the Problem

In order to solve the problem described above, a wearable device according to the present disclosure is a wearable device attached to a living body and includes a substrate that forms a first flow path, a second flow path, and a third flow path, a light source that is disposed on the substrate and emits light toward the second flow path, and a light receiving element that is disposed on the substrate to face the light source, receives the light emitted from the light source and transmitted through the second flow path, converts the received light into an electrical signal, and outputs the electrical signal, in which the first flow path includes one end that opens into a first side surface of the substrate and is configured to transport sweat secreted from skin of the living body, the second flow path has a diameter larger than a diameter of the first flow path, includes one end connected to another end of the first flow path, and is configured to transport the sweat, and the third flow path has a diameter smaller than the diameter of the second flow path, includes one end connected to another end of the second flow path and another end that opens into a second side surface of the substrate, and is configured to transport the sweat.

In order to solve the problem described above, a perspiration analysis apparatus according to the present disclosure includes the wearable device described above, a first calculation circuit that calculates, from a frequency of occurrence of a local maximum value or a local minimum value of the electrical signal output from the light receiving element, a physical amount related to perspiration of the living body, and an output unit configured to output the physical amount calculated and related to the perspiration.

In order to solve the problem described above, a perspiration analysis method according to the present disclosure includes causing a first flow path including one end that opens into a first side surface of a substrate to transport sweat secreted from skin of a living body, causing a second flow path having a diameter larger than a diameter of the first flow path and including one end connected to another end of the first flow path to transport the sweat, causing a third flow path having a diameter smaller than the diameter of the second flow path and including one end connected to another end of the second flow path and another end that opens into a second side surface of the substrate to transport the sweat, emitting light from a light source disposed on the substrate toward the second flow path, by a light receiving element disposed on the substrate to face the light source, receiving the light emitted from the light source and transmitted through the second flow path to convert the received light into an electrical signal and output the electrical signal, calculating, from the electrical signal output in the receiving, at least any of a physical amount related to perspiration of the living body and a concentration of a predetermined component included in the sweat, and outputting a calculation result in the calculating.

### Effects of Embodiments of the Invention

The present disclosure includes the first flow path including one end that opens into the first side surface of the substrate, the second flow path having a diameter larger than a diameter of the first flow path and including one end connected to another end of the first flow path, and the third flow path having a diameter smaller than the diameter of the second flow path and including one end connected to another end of the second flow path and another end that opens into the second side surface of the substrate. Thus, a physical amount related to sweat can be measured without using an air pump for replacing the air in a measurement system.

### BRIEF DESCRIPTION OF THE DRAWINGS

FIG. 1 is a schematic bottom view of a wearable device according to an embodiment of the present disclosure.

FIG. 2 is a cross-sectional view taken along a line II-II′ in FIG. 1.

FIG. 3 is a cross-sectional view taken along a line III-III′ in FIG. 1.

FIG. 4 is a diagram for describing an electrical signal acquired by the wearable device according to the present embodiment.

FIG. 5A is a diagram for describing a state of sweat in a flow path corresponding to the electrical signal in FIG. 4.

FIG. 5B is a diagram for describing a state of sweat in the flow path corresponding to the electrical signal in FIG. 4.

FIG. 5C is a diagram for describing a state of sweat in the flow path corresponding to the electrical signal in FIG. 4.

FIG. 6 is a block diagram illustrating a functional configuration of a perspiration analysis apparatus including the wearable device according to the present embodiment.

FIG. 7 is a block diagram illustrating an example of a hardware configuration of the perspiration analysis apparatus including the wearable device according to the present embodiment.

FIG. 8 is a flowchart for describing an operation of the perspiration analysis apparatus including the wearable device according to the present embodiment.

### DETAILED DESCRIPTION OF ILLUSTRATIVE EMBODIMENTS

Hereinafter, a preferred embodiment of the present disclosure will be described in detail with reference to FIGS. 1 to 8.

### Summary of Embodiments of the Invention

First, an outline of a wearable device 1 according to an embodiment of the present disclosure will be described with reference to FIG. 1.

FIG. 1 is a diagram schematically illustrating a bottom surface of the wearable device 1. The wearable device 1 is attached to a user (living body) and includes a substrate that forms a first flow path 12, a second flow path 13, and a third flow path 14 that transport sweat SW secreted from a sweat gland of skin SK of the user. A diameter of the first flow path 12 and a diameter of the third flow path 14 are smaller than a diameter of the second flow path 13. In the present embodiment, the sweat SW flowing into the first flow path 12 is transported from the first flow path 12 to the second flow path 13. Furthermore, the sweat SW is transported from the second flow path 13 to the third flow path 14 and is discharged from the third flow path 14 to the outside.

In the wearable device 1, a light source 15 and a light receiving element 16 are disposed on the substrate so as to face each other across the second flow path 13. In the present embodiment, the substrate includes a first substrate 10 and a second substrate 11 bonded to one another.

Configuration of Wearable Device

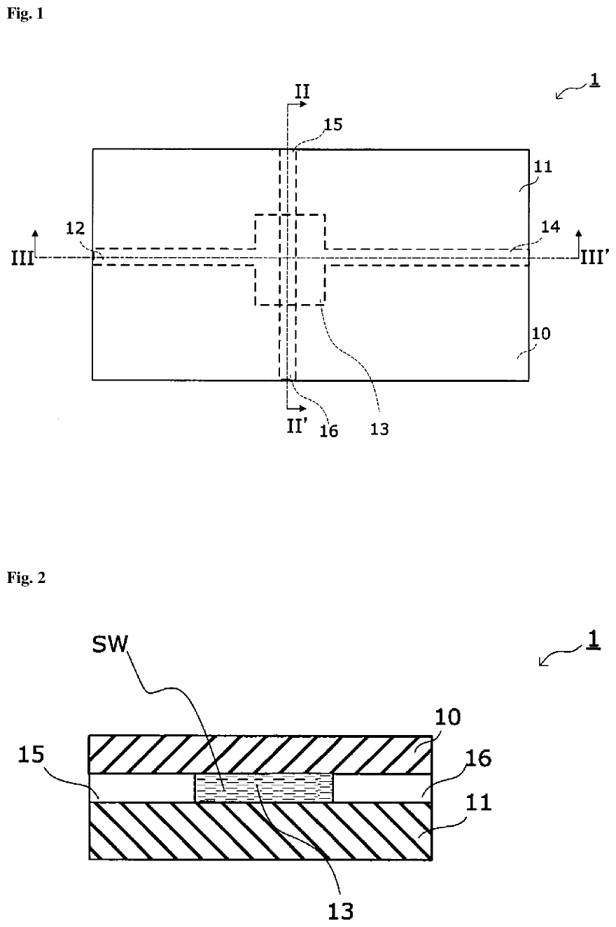

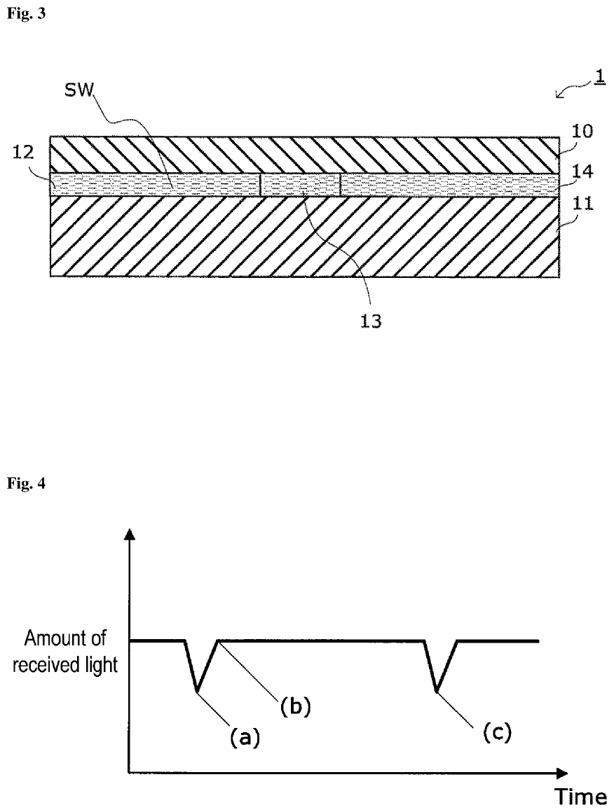

Next, the embodiment of the present disclosure will be described with reference to FIGS. 1 to 8. FIG. 1 is a schematic diagram of a bottom surface of the wearable device 1. FIG. 2 is a cross-sectional view of the wearable device 1 in FIG. 1 taken along a line II-II′. FIG. 3 is a cross-sectional view of the wearable device 1 in FIG. 1 taken along a line III-III′.

The wearable device 1 includes, for example, the first substrate 10 and the second substrate 11 that are attached to the user, the first flow path 12, the second flow path 13, the third flow path 14, the light source 15, and the light receiving element 16.

A surface of the first substrate 10 facing the second substrate 11 is bonded to the second substrate 11.

The second substrate 11 includes, in a surface facing the first substrate 10, a first groove, a second groove, and a third groove that form the first flow path 12, the second flow path 13, and the third flow path 14, respectively. In the present embodiment, the first flow path 12, the second flow path 13, and the third flow path 14 are formed by a space defined by bonding a surface of the second substrate 11 in which the grooves are formed and a surface of the first substrate 10 facing the surface of the second substrate 11.

Further, the light source 15 and the light receiving element 16 are disposed on the second substrate 11 so as to face each other across the second flow path 13.

Any insulating material can be used as a material of the first substrate 10 and the second substrate 11. For example, a hydrophilic material such as glass or a hydrophobic material such as resin may be used as the insulating material.

The first flow path 12 includes one end that opens into a side surface (first side surface) of the substrate acquired by bonding the first substrate 10 and the second substrate 11 to each other and the other end connected to one end of the second flow path 13, and transports the sweat SW secreted from the sweat gland of the skin SK. The first flow path 12 includes a space defined by the groove (first grooves) formed in the surface of the second substrate 11 facing the first substrate 10 and the first substrate 10.

An inlet structure (not illustrated) is provided at the one end of the first flow path 12 and collects the sweat SW. For example, the inlet structure that collects the sweat SW may be a flow path structure having an opening in contact with the skin SK of the user.

A cross-sectional shape of the first flow path 12 can be rectangular, circular, or the like. Further, the first flow path 12 is, for example, a thin tube having a certain flow path length and a certain flow path width, and a cross-sectional area can be, for example, approximately 1 mm², or 1 mm² or less. An inner wall of the first flow path 12 may be either hydrophilic or hydrophobic. Note that, even when the inner wall of the first flow path 12 is hydrophobic, the sweat SW secreted from the sweat gland is transported from the first flow path 12 to the second flow path 13 and, further to the third flow path 14 due to osmotic pressure of the sweat SW.

The second flow path 13 has a diameter larger than a diameter of the first flow path 12, includes one end connected to the other end of the first flow path 12, and transports the sweat SW. The other end of the second flow path 13 is connected to one end of the third flow path 14. The groove (second groove) of the second flow path 13 is formed in the surface of the second substrate 11 facing the first substrate 10. The second flow path 13 includes a space defined by the first substrate 10 and the second substrate 11 bonded to each other.

In the present embodiment, as illustrated in FIGS. 1 to 3, the second flow path 13 has a flow path width greater than a flow path length. Further, an inner wall of the second flow path 13 is hydrophobic. The second flow path 13 has volume that can retain at least one drop of droplets of the sweat SW. A cross-sectional shape of the second flow path 13 may be rectangular as illustrated in FIGS. 1 to 3 and may be circular or the like. Alternatively, the second flow path 13 may be formed as a spherical space in which a droplet fits.

Because the inner wall of the second flow path 13 is hydrophobic, when the sweat SW transported by the first flow path 12 is transported to an inlet of the second flow path 13, the sweat SW forms a droplet in the second flow path 13.

The third flow path 14 has a diameter smaller than a diameter of the second flow path 13, includes one end connected to the other end of the second flow path 13 and the other end that opens into a side surface (second side surface) of the substrate including the first substrate 10 and the second substrate 11 bonded to each other, and transports the sweat SW. The groove (third groove) of the third flow path 14 is formed in the surface of the second substrate 11 facing the first substrate 10. The third flow path 14 includes a space defined by the first substrate 10 and the second substrate 11 bonded to each other.

A cross-sectional shape of the third flow path 14 can be rectangular, circular, or the like. Further, the third flow path 14 is, for example, a thin tube having a certain flow path length and a certain flow path width and has a cross-sectional area of, for example, approximately 1 mm², or 1 mm² or less that is sufficiently smaller than a cross-sectional area of the second flow path 13. An inner wall of the third flow path 14 is hydrophilic. In the present embodiment, when a droplet of the sweat SW formed in the second flow path 13 comes into contact with an inlet (the one end) of the third flow path 14, the sweat SW having the volume of the formed droplet flows into the third flow path 14 so as to be sucked into the third flow path 14 and is transported to an outlet (the other end) of the third flow path 14.

The other end of the third flow path 14 may be provided with, for example, an outlet structure that facilitates discharge and evaporation of the sweat SW transported from the third flow path 14. As an example of the outlet structure provided at the other end of the third flow path 14, fibers such as cotton and silk, or a porous body such as a porous ceramic substrate can be used.

In this way, with the first flow path 12 being a thin tube, the second flow path 13 having a cross-sectional area of the flow path larger than those of the first flow path 12 and the third flow path 14 and including the hydrophobic inner wall, and the third flow path 14 including the hydrophilic inner wall and being a thin tube, the sweat SW is transported from the first flow path 12 to the second flow path 13, and further from the second flow path 13 to the third flow path 14 due to a capillary phenomenon.

For example, the light source 15 is disposed on the second substrate 11 and emits light toward the second flow path 13. The light source 15 is composed of a laser diode, for example. For example, as illustrated in FIGS. 1 and 2, the light source 15 is disposed so as to face the light receiving element 16 described below in a direction along a groove width across the groove that forms the second flow path 13 in the surface of the second substrate 11 facing the first substrate 10.

The light receiving element 16 is composed of a photodiode or the like and is disposed so as to face the light source 15 in the direction along the groove width across the groove that forms the second flow path 13 in the surface of the second substrate 11 facing the first substrate 10, for example. The light receiving element 16 receives light emitted from the light source 15 and transmitted through the second flow path 13 in which the sweat SW is transported. The light receiving element 16 converts the received light into an electrical signal and outputs the electrical signal. A light path from the light source 15 to the light receiving element 16 intersects the second flow path 13. For example, as illustrated in FIG. 1, a light path is formed along a vertical direction with respect to a flow path length in which the sweat SW is transported.

The wearable device 1 described above is manufactured by the following manufacturing method. First, a mold in which a groove being a flow path is formed by etching resin or Si is manufactured. Next, a metal structure is manufactured by electroforming based on the manufactured mold, and the manufactured mold is removed by etching or the like to acquire a metal mold. The metal mold is transferred to mold the second substrate 11 in which a flow path formed of a hydrophobic resin or the like is formed. Subsequently, the inner wall of the first flow path 12 and the third flow path 14 is subjected to surface treatment for making the inner wall hydrophilic by plasma treatment, for example. Finally, the surface of the first substrate 10 formed of an insulating material such as a hydrophobic resin and the surface of the second substrate 11 in which the groove being the flow path is formed are bonded together to acquire the wearable device 1.

The wearable device 1 can also use a hydrophilic insulating material, such as a glass substrate, as the first substrate 10 and the second substrate 11. In this case, the first flow path 12, the second flow path 13, and the third flow path 14 formed in the second substrate 11 have a hydrophilic inner wall. In this case, for the second flow path 13, the inner wall of the second flow path 13 is subjected to surface treatment for making the inner wall hydrophobic (water-repellent) by, for example, silane coupling treatment, fluorine plasma treatment, or the like. When fluorine plasma treatment is used, the inner wall becomes inert, and even when sebum or the like is included in the sweat SW, the inner wall of the second flow path 13 can be made water-repellent.

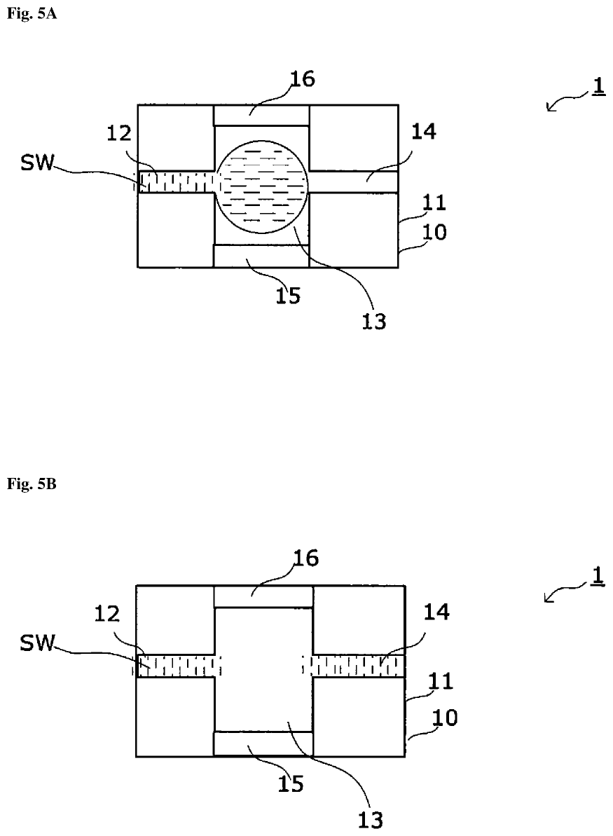

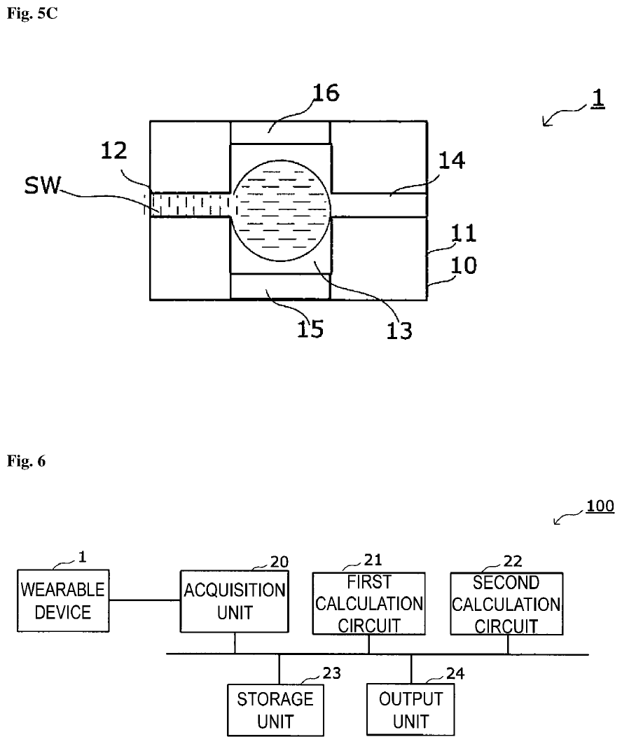

As illustrated in FIG. 5A, the sweat SW secreted from the sweat gland of the skin SK flows into the first flow path 12 and is transported to the second flow path 13. When an amount of perspiration further increases or perspiration further continues, the liquid sweat SW forms, for example, a droplet in the second flow path 13 including the hydrophobic inner wall. Before the droplet of the sweat SW is formed in the second flow path 13, light emitted from the light source 15 is transmitted through an air layer in the second flow path 13 and is received by the light receiving element 16. On the other hand, when the droplet is formed in the second flow path 13 and approaches the third flow path 14, light emitted from the light source 15 is transmitted through a liquid layer of the sweat SW and is received by the light receiving element 16.

Subsequently, as illustrated in FIG. 5B, by the amount of perspiration of the sweat SW further increasing or the perspiration further continuing, when the droplet in the second flow path 13 comes into contact with the inlet of the third flow path 14 including the hydrophilic inner wall, the sweat SW in the second flow path 13 is sucked into the third flow path 14 due to the capillary phenomenon. At this time, the droplet of the sweat SW in the second flow path 13 disappears, and thus the second flow path 13 has only the air layer, and light emitted from the light source 15 is transmitted through only the air layer and is received by the light receiving element 16.

Subsequently, as illustrated in FIG. 5C, when the amount of perspiration further increases or the perspiration further continues, a droplet of the sweat SW is formed again in the second flow path 13. In this way, a cycle of appearance and disappearance of the sweat SW in the second flow path 13 is repeated in accordance with a secretion rate (perspiration rate) of the sweat SW.

FIG. 4 is an example of an electrical signal indicating an amount of received light (light intensity) that is a physical amount related to the sweat SW optically measured by the wearable device 1 by the light source 15 and the light receiving element 16.

A vertical axis in FIG. 4 indicates the amount of light received by the light receiving element 16, and a horizontal axis indicates time. FIGS. 5A, 5B, and 5C illustrate states of the sweat SW flowing through the second flow path 13 at each time (a), (b), and (c) in FIG. 4, respectively.

As illustrated in FIG. 4, an electrical signal output from the light receiving element 16 has a signal waveform such as a continuous pulse waveform in accordance with the cycle of formation and disappearance of a droplet of the sweat SW in the second flow path 13.

As illustrated in FIG. 5A, the current value at the time (a) in FIG. 4 indicates that a droplet of the sweat SW is formed in the second flow path 13 and intersects the light path from the light source 15 to the light receiving element 16. An amount of received light (light intensity) of light transmitted through the liquid layer of the sweat SW is reduced further than that when the air is included as a medium in the second flow path 13. Further, the amount of received light changes in accordance with an amount of the sweat SW transported to the second flow path 13, i.e., an amount of the air layer and the sweat SW that are included in the medium.

At the time (b) in FIG. 4, as illustrated in FIG. 5B, the light emitted from the light source 15 is received by the light receiving element 16 through only the air layer of the second flow path 13. Subsequently, when the amount of perspiration occurs for a certain period of time, as illustrated in FIG. 5C corresponding to the time (c) in FIG. 4, the sweat SW is transported again to the second flow path 13, the light from the light source 15 is transmitted while the medium changes in an order from the air layer to the liquid layer of the sweat SW, and the light is received by the light receiving element 16.

Functional Blocks of Perspiration Analysis Apparatus

Next, a functional configuration of a perspiration analysis apparatus 100 including the wearable device 1 described above will be described with reference to a block diagram in FIG. 6.

The perspiration analysis apparatus 100 includes the wearable device 1, an acquisition unit 20, a first calculation circuit 21, a second calculation circuit 22, a storage unit 23, and an output unit 24.

The acquisition unit 20 acquires an electrical signal acquired by the wearable device 1. The acquisition unit 20 performs signal processing such as amplification, noise removal, and AD conversion of the acquired electrical signal. Time-series data of the acquired electrical signal is accumulated in the storage unit 23. As illustrated in FIG. 4, for example, the time-series data of the electrical signal acquired by the acquisition unit 20 is a waveform having a peak in accordance with the cycle of the appearance and disappearance of the sweat SW in the second flow path 13 described above.

The first calculation circuit 21 calculates a physical amount related to perspiration from a frequency of occurrence of a local maximum value or a local minimum value of the electrical signal. For example, the first calculation circuit 21 calculates, from the time-series data of the electrical signal, an amount of perspiration by multiplying predetermined volume of a droplet of the sweat SW formed in the second flow path 13 by the number of times of appearance (or disappearance) of the droplet (i.e., the number of peaks in FIG. 4). The volume of the droplet of the sweat SW can be previously obtained by calculation from the volume of the second flow path 13 and characteristics of the sweat SW, for example. Alternatively, the volume of the droplet may be obtained by experiment.

Further, the first calculation circuit 21 calculates a perspiration rate per unit area by dividing predetermined volume of a droplet of the sweat SW by a cycle of appearance (or disappearance) of the sweat SW in the second flow path 13 and an area of the skin SK of the user in contact with the first flow path 12 or the inlet structure connected to the first flow path 12. Note that a cross-sectional area of the first flow path 12 or a cross-sectional area of an opening of the inlet structure can be used as an area of the skin SK.

The second calculation circuit 22 calculates a concentration of a predetermined component included in the sweat SW from the electrical signal acquired by the wearable device 1. For example, the second calculation circuit 22 calculates a concentration of a component (water, sodium chloride, urea, lactic acid, and the like) included in the sweat SW. More specifically, with a laser wavelength of the light source 15 as an absorption wavelength of a specific component of the sweat SW, the second calculation circuit 22 can calculate a specific component concentration of the sweat from the amount of light received by the light receiving element 16 when the sweat SW is transported to the second flow path 13.

The storage unit 23 stores time-series data of the electrical signal acquired from the wearable device 1 by the acquisition unit 20. In the storage unit 23, information related to volume of the second flow path 13 and a laser wavelength of the light source 15 is stored in advance.

The output unit 24 outputs the amount of perspiration, the perspiration rate, and the component concentration of the sweat SW calculated by the first calculation circuit 21 and the second calculation circuit 22. The output unit 24 can display a calculation result on a display device (not illustrated), for example. Alternatively, the output unit 24 may send a calculation result to an external communication terminal device (not illustrated) by a communication I/F 105 described below.

Hardware Configuration of Perspiration Analysis Apparatus

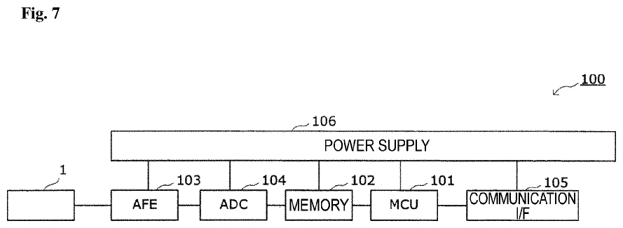

Next, an example of a hardware configuration that implements the perspiration analysis apparatus 100 including the wearable device 1 having the above-described functions will be described with reference to FIG. 7.

As illustrated in FIG. 7, for example, the perspiration analysis apparatus 100 can be implemented by a computer including an MCU 101, a memory 102, an AFL 103, an ADC 104, and a communication I/F 105 connected to each other through a bus, and a program for controlling these hardware resources. In the perspiration analysis apparatus 100, for example, the wearable device 1 provided outside is connected through the bus. Further, the perspiration analysis apparatus 100 includes a power supply 106 and supplies power to the entire device other than the wearable device 1 illustrated in FIGS. 6 and 7.

A program causing the micro control unit (MCU) 101 to perform various controls or calculations is previously stored in the memory 102. Each function of the perspiration analysis apparatus 100 including the acquisition unit 20, the first calculation circuit 21, and the second calculation circuit 22 illustrated in FIG. 6 is implemented by the MCU 101 and the memory 102.

The analog front end (AFE) 103 is a circuit that amplifies a measurement signal that is a weak electrical signal representing an analog current value measured by the wearable device 1.

The analog-to-digital converter (ADC) 104 is a circuit that converts an analog signal amplified by the AFE 103 into a digital signal at a predetermined sampling frequency. The AFE 103 and the ADC 104 implement the acquisition unit 20 in FIG. 6.

The memory 102 is implemented by a non-volatile memory such as a flash memory, a volatile memory such as a DRAM, and the like. The memory 102 temporarily stores time-series data of signals output from the ADC 104. The memory 102 implements the storage unit 23 in FIG. 6.

The memory 102 includes a program storage area in which a program used by the perspiration analysis apparatus 100 to perform perspiration analysis processing is stored. Further, for example, it may have a backup area for backing up the above-described data, programs, or the like.

The communication I/F 105 is an interface circuit for communicating with various external electronic devices through a communication network NW.

For example, a communication interface compatible with a wired or wireless data communication standard such as LTE, 3G, 4G, 5G, Bluetooth (trade name), Bluetooth Low Energy, and Ethernet (trade name) and an antenna are used as the communication I/F 105. The output unit 24 in FIG. 6 is implemented by the communication I/F 105.

Note that the perspiration analysis apparatus 100 acquires time information from a clock incorporated in the MCU 101 or a time server (not illustrated) and uses the time information as sampling time.

Perspiration Analysis Method

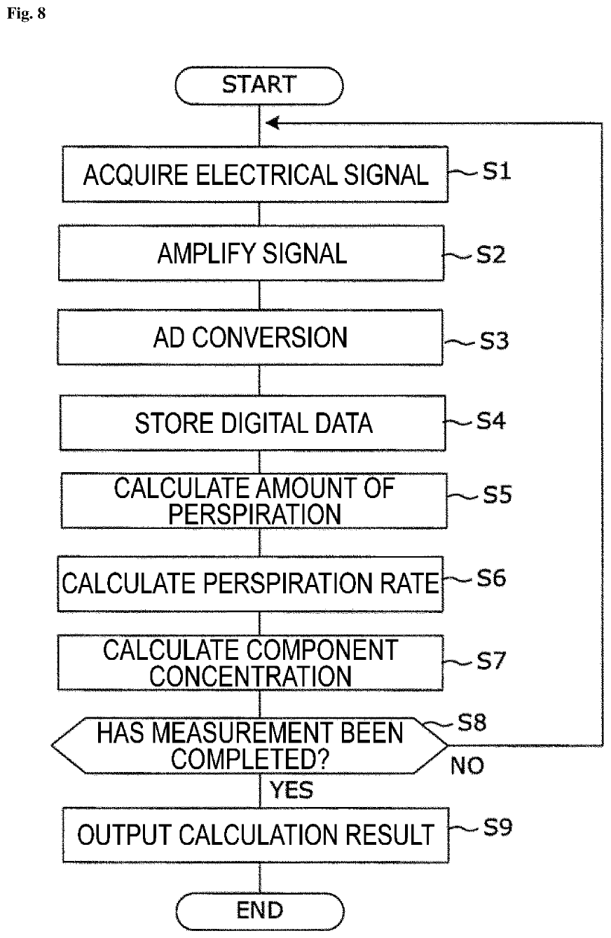

Next, an operation of the perspiration analysis apparatus 100 including the wearable device 1 having the above-described configuration will be described with reference to a flowchart in FIG. 8. When the wearable device 1 is previously attached to the user, the power supply 106 is turned on, and the perspiration analysis apparatus 100 is activated, the following processing operations are performed.

First, the acquisition unit 20 acquires an electrical signal indicating a current value from the wearable device 1 (step S1). Next, the acquisition unit 20 amplifies the electrical signal (step S2). More specifically, the AFE 103 amplifies a weak current signal measured by the wearable device 1.

Next, the acquisition unit 20 performs AD conversion on the electrical signal amplified in step S2 (step S3). Specifically, the ADC 104 converts an analog signal amplified by the AFE 103 into a digital signal at a predetermined sampling frequency. Time-series data of the electrical signal converted into the digital signal is stored in the storage unit 23 (step S4).

Next, the first calculation circuit 21 calculates an amount of perspiration of the user from the acquired electrical signal (step S1). Subsequently, the first calculation circuit 21 calculates a perspiration rate from the electrical signal (step S6).

Next, the second calculation circuit 22 calculates a concentration of a predetermined component included in the sweat SW from the acquired electrical signal (step S7). Subsequently, when the measurement has been completed (step S8: YES), the output unit 24 outputs a calculation result including the amount of perspiration, the perspiration rate, and the component concentration (step S9). On the other hand, when the measurement has not been completed (step S8: NO), the processing returns to step S1.

Note that the first calculation circuit 21 may be configured to calculate either the amount of perspiration or the perspiration rate. The first calculation circuit 21 can also be configured, by setting, to calculate any one or two values of the amount of perspiration, the perspiration rate, and the component concentration, and an order in which the values are calculated is optional.

Further, in the described embodiment, the wearable device 1 may be fixed to a body of a user by a band or may be fixed to clothing worn by the user as long as the wearable device 1 is connected to the inlet structure that collects the sweat SW from the skin SK of the user.

As described above, according to the present embodiment, the wearable device 1 transports the sweat SW by the second flow path 13 that is formed in the substrate and includes the hydrophobic inner wall and by the third flow path 14 that is connected to the second flow path 13, has a diameter smaller than a diameter of the second flow path 13, and includes the hydrophilic inner wall. Further, the light source 15 and the light receiving element 16 are disposed so as to face each other across the second flow path 13. Thus, the wearable device 1 can measure a physical amount related to sweat without using an air pump. Further, the wearable device 1 can measure, from the measured physical amount related to the sweat, a physical amount related to perspiration such as an amount of perspiration and a perspiration rate, and a component included in the sweat.

The wearable device 1 according to the present embodiment collects the sweat SW in a liquid state without using an air pump and transports the sweat SW from the second flow path 13 to the third flow path 14 for each certain volume, and thus the size of the wearable device 1 can be made smaller. Further, as a result, the size of the perspiration analysis apparatus 100 can be reduced.

Further, the wearable device 1 according to the present embodiment includes the light source 15 and the light receiving element 16 and measures time-series data of a current signal due to a change in amount of received light in accordance with a cycle in which the sweat SW appears in the second flow path 13 and is transported to the third flow path 14 at a certain cycle. Thus, the wearable device 1 attached to a user can optically measure a physical amount related to sweat.

Although the embodiments of the wearable device, the perspiration analysis apparatus, and the perspiration analysis method according to the present disclosure have been described above, the present disclosure is not limited to the above-described embodiments and can be modified into various forms that can be conceived by a person skilled in the art within the scope of the disclosure described in the aspects.

### REFERENCE SIGNS LIST

1 Wearable device
10 First substrate
11 Second substrate
12 First flow path
13 Second flow path
14 Third flow path
15 Light source
16 Light receiving element
20 Acquisition unit
21 First calculation circuit
22 Second calculation circuit
23 Storage unit
24 Output unit
100 Perspiration analysis apparatus
101 MCU
102 Memory
103 AFE
104 ADC
105 Communication I/F
106 Power supply
SW Sweat.

1-8. (canceled)

9. A wearable device configured to be attached to a living body, the wearable device comprising:
a substrate that comprises a first flow path, a second flow path, and a third flow path;
a light source on the substrate and configured to emit light toward the second flow path; and
a light receiving element on the substrate to face the light source and configured to receive the light emitted from the light source and transmitted through the second flow path, convert the received light into an electrical signal, and output the electrical signal,
wherein the first flow path includes a first end that opens onto a first side surface of the substrate and is configured to transport sweat secreted from skin of the living body,
wherein the second flow path has a diameter larger than a diameter of the first flow path, includes a first end connected to a second end of the first flow path, and is configured to transport the sweat, and
wherein the third flow path has a diameter smaller than the diameter of the second flow path, includes a first end connected to a second end of the second flow path and a second end that opens into a second side surface of the substrate, and is configured to transport the sweat.

10. The wearable device according to claim 9, wherein:
an inner wall of the second flow path is hydrophobic; and
an inner wall of the third flow path is hydrophilic.

11. The wearable device according to claim 9, wherein a light path from the light source to the light receiving element intersects the second flow path.

12. The wearable device according to claim 9, wherein:
the substrate includes a first substrate and a second substrate bonded to each other;
the second substrate includes, in a surface facing the first substrate, a first groove, a second groove, and a third groove that form, together with the first substrate, the first flow path, the second flow path, and the third flow path, respectively; and
the light source and the light receiving element are disposed to face each other in a direction along a groove width across the second groove that forms the second flow path in the surface of the second substrate facing the first substrate.

13. The wearable device according to claim 9, wherein the second flow path has a flow path width greater than a flow path length.

14. A perspiration analysis apparatus, comprising:
a wearable device configured to be attached to a living body, the wearable device comprising:
a substrate that comprises a first flow path, a second flow path, and a third flow path;
a light source on the substrate and configured to emit light toward the second flow path; and
a light receiving element on the substrate to face the light source and configured to receive the light emitted from the light source and transmitted through the second flow path, convert the received light into an electrical signal, and output the electrical signal, wherein the first flow path includes a first end that opens onto a first side surface of the substrate and is configured to transport sweat secreted from skin of the living body, wherein the second flow path has a diameter larger than a diameter of the first flow path, includes a first end connected to a second end of the first flow path, and is configured to transport the sweat, and wherein the third flow path has a diameter smaller than the diameter of the second flow path, includes a first end connected to a second end of the second flow path and a second end that opens into a second side surface of the substrate, and is configured to transport the sweat;
a first calculation circuit configured to calculate, from a frequency of occurrence of a local maximum value or a local minimum value of the electrical signal output from the light receiving element, a physical amount related to perspiration of the living body; and
an output device configured to output the physical amount calculated and related to the perspiration.

15. The perspiration analysis apparatus according to claim 14, further comprising:
a second calculation circuit configured to calculate, from the electrical signal output from the light receiving element, a concentration of a predetermined component included in the sweat, wherein the output device is configured to output the concentration calculated by the second calculation circuit.

16. The perspiration analysis apparatus according to claim 15, wherein the concentration of the predetermined component is an electrolyte concentration.

17. The perspiration analysis apparatus according to claim 14, wherein:
an inner wall of the second flow path is hydrophobic; and
an inner wall of the third flow path is hydrophilic.

18. The perspiration analysis apparatus according to claim 14, wherein a light path from the light source to the light receiving element intersects the second flow path.

19. The perspiration analysis apparatus according to claim 14, wherein:
the substrate includes a first substrate and a second substrate bonded to each other;
the second substrate includes, in a surface facing the first substrate, a first groove, a second groove, and a third groove that form, together with the first substrate, the first flow path, the second flow path, and the third flow path, respectively; and
the light source and the light receiving element are disposed to face each other in a direction along a groove width across the second groove that forms the second flow path in the surface of the second substrate facing the first substrate.

20. The perspiration analysis apparatus according to claim 14, wherein the second flow path has a flow path width greater than a flow path length.

21. A perspiration analysis method, comprising:
transporting, by a first flow path, sweat secreted from skin of a living body, the first flow path including a first end that opens onto a first side surface of a substrate;
transporting, by a second flow path, the sweat, the second flow path having a diameter larger than a diameter of the first flow path and including a first end connected to a second end of the first flow path;
transporting, by a third flow path, the sweat, the third flow path having a diameter smaller than the diameter of the second flow path and including a first end connected to a second end of the second flow path and a second end that opens onto a second side surface of the substrate;
emitting light from a light source toward the second flow path, the light source being disposed on the substrate;
receiving, by a light receiving element disposed on the substrate to face the light source, the light emitted from the light source and transmitted through the second flow path to convert the light into an electrical signal and output the electrical signal;
calculating, from the electrical signal, a physical amount related to perspiration of the living body or a concentration of a predetermined component included in the sweat; and
outputting a calculation result in the calculating.

22. The perspiration analysis method according to claim 21, wherein the concentration of the predetermined component is an electrolyte concentration.

23. The perspiration analysis method according to claim 21, wherein:
an inner wall of the second flow path is hydrophobic; and
an inner wall of the third flow path is hydrophilic.

24. The perspiration analysis method according to claim 21, wherein a light path from the light source to the light receiving element intersects the second flow path.

25. The perspiration analysis method according to claim 21, wherein:
the substrate includes a first substrate and a second substrate bonded to each other;
the second substrate includes, in a surface facing the first substrate, a first groove, a second groove, and a third groove that form, together with the first substrate, the first flow path, the second flow path, and the third flow path, respectively; and
the light source and the light receiving element are disposed to face each other in a direction along a groove width across the second groove that forms the second flow path in the surface of the second substrate facing the first substrate.

26. The perspiration analysis method according to claim 21, wherein the second flow path has a flow path width greater than a flow path length.
